(19) United States
(12) Patent Application Publication
(10) Pub. No.: US 2006/0121800 A1
(43) Pub. Date: Jun. 8, 2006

(54) Connecting lead for a sensor

(76) Inventors: Helmut Weyl, Wiesbaden (DE); Juergen Werner, Leinfelden-Echterdingen (DE); Andreas pesch, Krefeld (DE); Gerald West, Weissach-Flacht (DE); Andreas Werner, Korntal-Muenchingen (DE); Hermann Brauer, Barssel (DE); Tanil Gezgin, Ovelgonne (DE); Horst Kontants, Oldenburg (DE); Manfred Knoetig, Nordenham (DE)

Correspondence Address: KENYON & KENYON LLP, ONE BROADWAY, NEW YORK, NY, 10004, US

(21) Appl. No.: 10/526,150

(22) PCT Filed: Jul. 14, 2003
(86) PCT No.: PCT/DE03/02360

§ 371 Date: Dec. 12, 2005

(30) Foreign Application Priority Data
Aug. 31, 2002 (DE) 102 40 238.8

(51) Int. Cl. G01N 33/497

(52) U.S. Cl. 439/875; 73/23.31

(57) ABSTRACT

A connecting lead for a sensor, in particular for a sensor for determining a physical property of a measured gas, e.g. for determining the temperature or oxygen content in the exhaust gas of internal combustion engines, is described, said sensor comprising a sheathing tube, at least two electrical conductors extending in the sheathing tube, and an insulating arrangement that insulates the electrical conductors from one another and from the sheathing tube. To allow the use of unsheathed bare metal wires as electrical conductors, and avoid short circuits on the one hand between the metal wires and on the other hand between the metal wires and the sheathing tube, especially upon bending of the metal tube during installation, the insulating arrangement has a plurality of insulating elements, braced against one another, that have at least two through holes through each of which one electrical conductor is guided.

### FIELD OF THE INVENTION

The invention relates to a connecting lead for a sensor, in particular for a sensor for determining a physical property of a measured gas, in particular for determining the oxygen content or temperature in the exhaust gas of internal combustion engines.

### BACKGROUND INFORMATION

In sensors that are used as exhaust gas lambda probes in the exhaust gas fittings of internal combustion engines in motor vehicles, upon installation the sheathing tube is bent largely at a right angle in order to make contact with the connecting lead, i.e. to allow connection to the electrical system of the motor vehicle. In order reliably to rule out a short-circuit of the electrical conductors, the electrical conductors are electrically insulated from one another and from the sheathing tube.

In a known connecting lead for a sensor of this kind (German Patent No. 195 23 911), the electrical conductors are sheathed in a high-strength electrical insulation, e.g. glass filament, and four or five sheathed electrical conductors are received, with the highest possible packing density, in a sheathing tube made of a temperature-resistant metal, e.g. CrNi or NiCr alloys. At the connection end, the electrical conductors are welded onto crimp sleeves in which the ends of connecting cables leading to a connector plug are caulked. The crimp sleeves are encapsulated, together with one end of the sheathing tube and the end region of the connecting cable, in a sealing element made, for example, of PTFE. To allow the sheathing tube to be bent without damage, care must be taken that the sheathed electrical conductors have sufficient looseness within the sheathing tube to compensate for the changes, during bending of the metal tube, in the lengths of the electrical conductors inside the sheathing tube.

In a heat-resistant connecting lead for an exhaust gas lambda probe that is also known (European Published Patent Application No. 0 843 321), a pair of bare electrical conductors made of nickel wire, and a pair of aeration tubes made of stainless steel, extend inside the stainless-steel sheathing tube. The electrical insulation is made up of a magnesium powder that is introduced into the metal tube in such a way that the two pairs of electrical conductors and aeration tubes are disposed, diametrically opposite one another, at the four corners of a square, and are completely insulated by the magnesium powder from one another and from the sheathing tube. A connecting lead of this kind cannot be bent upon installation.

### SUMMARY OF THE INVENTION

The connecting lead according to the present invention has the advantage that the electrical conductors are guided by the insulating disks at a defined spacing from one another and from the sheathing tube, and bare wires can therefore be used as electrical conductors, without the sheathing (made of high-temperature-resistant material) that is very expensive to manufacture. The process of manufacturing the connecting lead can be configured in very simple and inexpensive fashion, since the insulating elements merely need to be threaded onto the conductors, and the threaded-on unit can then easily be pulled into the sheathing tube.

According to a preferred embodiment of the invention, the insulating elements are braced directly against one another in one element subregion and have, in the other element subregion remaining in the bracing plane, a clearance from one another that increases toward the element periphery. This spacing can be achieved by beveling or rounding the insulating elements. This geometry of the insulating elements guarantees the bendability of the connecting lead, since as the sheathing tube is bent, the insulating elements can assume an acute-angle incidence to one another because of the space present in the subregion, and thus allow curved guidance of the sheathing tube. As the sheathing tube is bent, the distances between the electrical conductors on the one hand, and between the electrical conductors and the sheathing tube on the other hand, are kept constant, and a short-circuit due to contact between the bare wires is avoided.

According to a preferred embodiment of the invention, the insulating elements are embodied as disks whose at least one disk surface are beveled toward the disk center in one subregion, and rest against one another with their flat disk surface region. The partial beveling of the insulating disks can be performed on each disk surface, or on one of the two disk surfaces. Instead of a bevel, a rounding can also be performed in such a way that a rounding radius joins the one disk surface to the other.

According to an advantageous embodiment of the invention, the through holes in each insulating disk are disposed in such a way that their hole axes lie next to one another on one diameter line. As a result, all the electrical conductors extend in a neutral zone of the sheathing tube, so that their lengths, clamped in place at the tube ends, are not modified upon bending.

According to an advantageous embodiment of the invention, the insulating disks each have a through opening, the through openings in the insulating disks resting against one another being mutually aligned. Guided through the through holes is a preferably round spring rod that is retained in axially nondisplaceable fashion in the sheathing tube. Retention is accomplished by axial bracing of the spring rod in the region of the tube ends. The spring rod places the insulating disks under stress after the sheathing tube has been bent, so that vibrations of the insulating disks during vehicle operation, which might cause breakage of the insulating disks, are prevented.

According to a preferred embodiment of the invention, the two outer ones of the insulating disks lying against one another are braced axially in the sheathing tube. The bracing is accomplished at the connection end of the sheathing tube by a seal element made of electrically insulating material and pressed into the sheathing tube, and bracing at the sensor end of the sheathing tube is accomplished by an insulating element that braces against the sheathing tube. The insulating element is in turn braced against at least one end disk, made of electrically insulating material, that closes off the sensor end of the sheathing tube. The insulating element and the at least one end disk are located in that part of the sheathing tube that is not bent but remains straight. The at least one end disk defines the desired connection pattern of the electrical conductors for the sensor element; and the insulating element creates, with its through orifices, the transition from the disposition, which deviates spatially from the connection pattern, of the through holes for the electrical conductors in the insulating disks.

### BRIEF DESCRIPTION OF THE DRAWINGS

FIG. 1 is a longitudinal section of a connecting lead for a sensor, in the state as delivered.

FIG. 2 is a side view, partially sectioned, of the connecting lead in FIG. 1 after final installation.

FIG. 3 is a side view of an insulating disk in the connecting lead in FIGS. 1 and 2.

FIG. 4 is a plan view of the insulating disk in the direction of arrow IV in FIG. 3.

FIG. 5 is a perspective view of the insulating disk in FIGS. 3 and 4.

FIG. 6 is a side view of an insulating element in the connecting lead in FIGS. 1 and 2.

FIG. 7 is a plan view of the insulating element in the direction of arrow VII in FIG. 6.

FIG. 8 is a perspective depiction of the insulating element in FIGS. 6 and 7.

FIG. 9 is a side view of an end disk of the connecting lead in FIGS. 1 and 2.

FIG. 10 is a plan view of the end disk in the direction of arrow X in FIG. 9.

FIG. 11 is a perspective depiction of the end disk in FIGS. 9 and 10.

### DETAILED DESCRIPTION

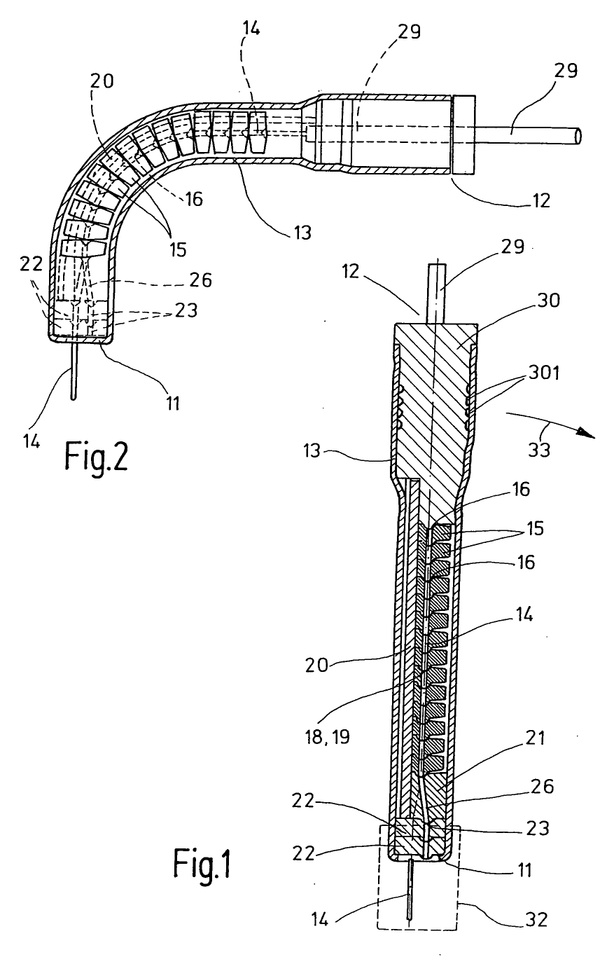

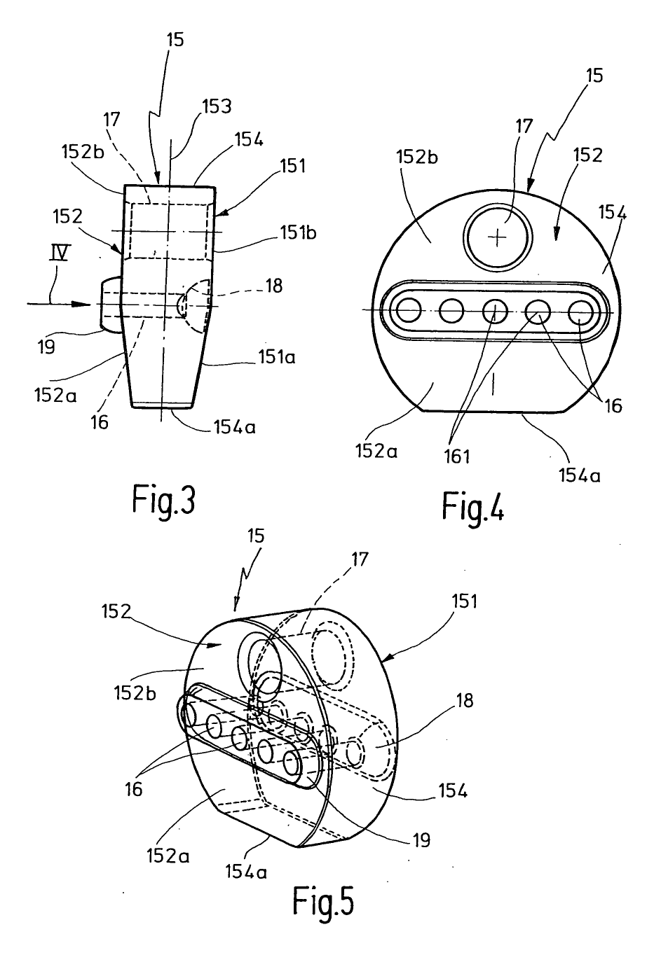

The connecting lead depicted in FIGS. 1 and 2 for a sensor, in particular for a sensor for determining a physical property of a measured gas, such as the temperature or oxygen concentration in the exhaust gas of internal combustion engines of motor vehicles, serves to join the sensor element (not depicted here), exposed to the measured exhaust gas, to a connector plug (not depicted here) for connecting the sensor to a control unit in the electrical system of the motor vehicle. Connecting lead 11 has a sheathing tube 13 made of high-temperature-resistant metal and, in the exemplary embodiment, a total of five electrical conductors 14 that extend in the interior of sheathing tube 13 between a sensor end 11 and a connector end 12 of sheathing tube 13. Electrical conductors 14 are embodied as bare, high-temperature-resistant wires. In order to avoid short circuits on the one hand between electrical conductors 14 and on the other hand between electrical conductors 14 and sheathing tube 13, electrical conductors 14 are guided in insulating means that prevent electrical conductors 14 from coming into mutual contact or into contact with sheathing tube 13 even in the context of a bending of sheathing tube 13 that occurs during installation, as depicted in FIG. 2. Provided for this purpose are a plurality of insulating elements, braced against one another, that are embodied in the exemplary embodiment as insulating disks 15 but can also have a different geometric shape. Insulating disks 15 rest with their disk surfaces 151, 152 (FIG. 3) against one another and are partially braced with their peripheral surfaces 154 (FIG. 3) against sheathing tube 13. Insulating disks 15 have mutually aligned through holes 16 (FIG. 3), and one of electrical conductors 14 is guided through each mutually aligned through hole 16.

FIGS. 3 through 5 depict an insulating disk 15 in a side view, plan view, and perspective view. The two mutually parallel disk surfaces 151, 152 are beveled at an acute angle toward disk center 153 in the lower surface region so that there results on each disk surface 151, 152, as is evident in FIG. 1, a region that extends parallel to the disk center, hereinafter referred to as parallel surface 151b, 152b, and a region proceeding at an obtuse angle therefrom, hereinafter called oblique surface 151a, 152a. The mutually facing oblique surfaces 151a, 152a of two adjacent insulating disks 15 enclose an acute angle between them, whereas parallel surfaces 151b and 152b rest in planar fashion against one another. Each insulating disk 15 rests with its peripheral surface 154 against the inner wall of sheathing tube 13. Peripheral surface 154 possesses a flat surface portion 154a extending in chord-like fashion. Placed on one diameter line extending parallel to this flat surface portion 154a are hole axes 161 of five equidistantly disposed through holes 16. Their number corresponds to the number of electrical conductors 14 to be guided in sheathing tube 13, that number being arbitrary and depending on the connection requirements of the sensor element. At a radial distance from this diameter line, a round through opening 17 is introduced in the region of parallel surfaces 151b, 152b. As is evident from FIGS. 3 and 5 and also from FIGS. 1 and 2, each insulating disk 15 has a concave indentation 18 on disk surface 151 and a convex protrusion 19 on disk surface 152. Indentation 19 and protrusion 19 respectively surround the entrance openings and exit openings of through holes 16. Indentations 18 and protrusions 19 are matched to one another in terms of shape in such a way that indentations 18 and protrusions 19 of insulating disks 15 that rest against one another engage conformingly into one another (cf. FIGS. 1 and 2).

As is evident from FIGS. 1 and 2, in the exemplary embodiment of the connecting lead described here, a total of fourteen insulating disks 15 are serially arranged and retained in axially nondisplaceable fashion in sheathing tube 13 in the manner described. The number of insulating disks 15 depends on the length of sheathing tube 13. A round spring rod 20 is guided through the mutually aligned through openings 17 and is likewise retained nondisplaceably in the axial direction in sheathing tube 13. One of the five electrical conductors 14, of which only one is visible in FIGS. 1 and 2, is guided through each of the mutually aligned through holes 16 in insulating disks 15.

Disposed at sensor end 11 of measurement tube 13, specifically in the portion of sheathing tube 13 that is not bent during installation but remains straight, are an insulating element 21 and two end disks 22, resting against one another, that constitute the sensor-end bracing for the series of insulating disks 15. Sheathing tube 13 is crimped over at the end onto the outer end disk 22.

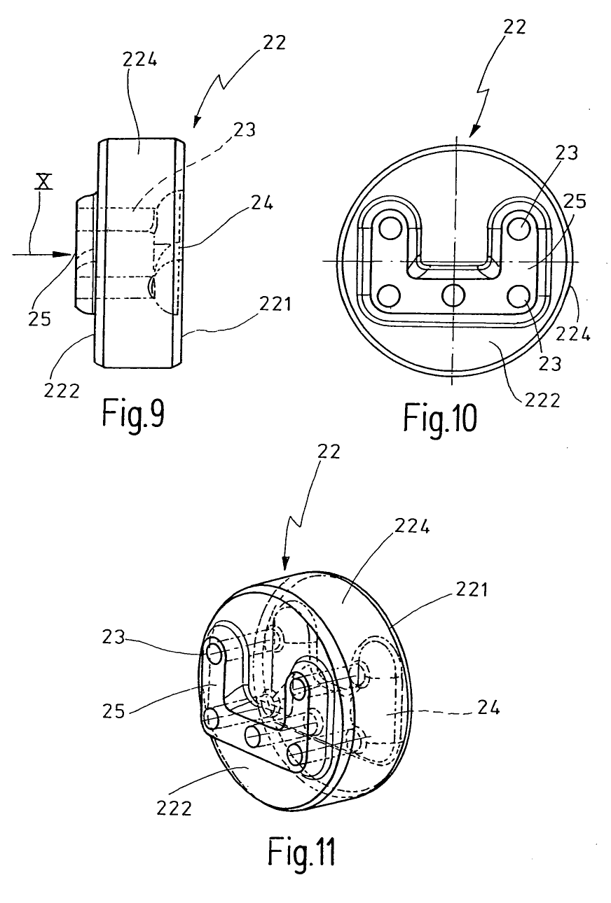

FIGS. 9 through 11 show an enlarged depiction of end disk 22. It is round in shape, and is braced with its peripheral surface 224 against the inner wall of sheathing tube 13. It possesses five through holes 23, corresponding to the number of electrical conductors 14 and having the same diameter as through holes 16 in insulating disks 15, and are disposed in accordance with the connection pattern of electrical conductors 14 defined by the sensor element. In the exemplary embodiment of FIGS. 9 through 11, the connection pattern is approximately U-shaped, three through holes 23 being located in the crosspiece of the U and one through hole 23 in each limb of the U. A different connection pattern is of course possible, for example with three through holes 23 lying on one of two parallel lines that are equidistant from the diameter line. Disk surfaces 221 and 222 of end disk 22 are flat and parallel to one another. Once again a concave indentation 24 is present on disk surface 221, and a geometrically identical convex protrusion 25 on disk surface 222, each respectively surrounding the entrance openings and exit openings of through holes 23.

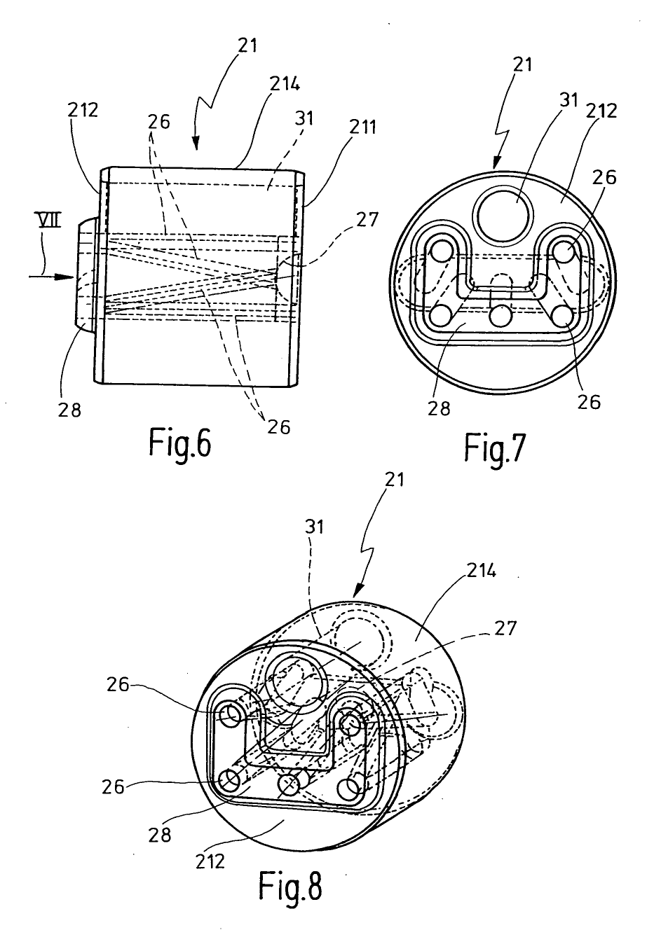

Insulating element 21, made of high-temperature-resistant electrical insulating material, is depicted in FIGS. 6 through 8. Through holes 26 are introduced into insulating element 21 in such a way that their entrance openings, located in end surface 211 of insulating element 21, are located congruently with the exit openings on disk surfaces 152 of insulating disks 15, and their exit openings disposed on end surface 212 are located congruently with the hole pattern of through holes 23 in end disk 22. In addition, an axial through orifice 31 is introduced into insulating element 21 in such a way that it aligns with through openings 17 in insulating disks 15. Axial through orifice 31 has a diameter identical to that of through openings 17, and serves for the passage of spring rod 20. A concave indentation 27 is once again recessed into end surface 211 of insulating element 21 in such a way that it can conformingly receive convex protrusion 19 of an insulating disk 15. Projecting on end surface 212 is a convex protrusion 28 which is configured so that it is conformingly insertable into concave indentation 24 of an end disk 22.

Near connection end 12 of sheathing tube 13, electrical conductors 14 are each joined to an electrical connecting cable 29 by ultrasonic welding. Connecting cables 29, of which only one is visible in FIGS. 1 and 2, are connected to a connector plug (not depicted here). At this connector end 12 of sheathing tube 13, the series of insulating disks 15 is braced by a seal element 30 pressed into end 12 of sheathing tube 13. This seal element 30 has on its periphery circumferential sealing lips 301, axially spaced apart from one another, that press against the inner wall of sheathing tube 13 and ensure a sufficient sealing effect. Spring rod 20, guided through the through openings 17 in insulating disks 15 and through axial through orifice 31 in insulating element 21, is braced at one end against seal element 30 and at the other end against end disk 22 resting against insulating element 21.

Upon assembly of the connecting lead, the individual electrical conductors 14 are threaded through the mutually aligned through holes 16 in insulating disks 15, through the through orifices 26 in insulating element 21, and through the through holes 23 in the two end disks 22, and protrude at sensor end 11 of sheathing tube 13 so that contact can be appropriately made to them from the sensor element. A protective cap 32, indicated with dashed lines in FIG. 1, can be slid onto sensor end 11 of sheathing tube 13 as a transport protector, protecting the protruding ends of electrical conductors 14 from damage. At connector end 12 of sheathing tube 13, seal element 30, which surrounds the connector ends of electrical conductors 14 and the connecting cables 29 contacted thereto, is pressed into sheathing tube 13; sheathing tube 13 is then rolled over in this region so that a positive and nonpositive join is produced between sheathing tube 13 and seal element 30.

Upon installation of the sensor, the connecting lead is bent at a right angle in the direction of arrow 33 in FIG. 1 so that it assumes the shape depicted in FIG. 2. This bending is possible because of the geometry of insulating disks 15 described above, since the latter are fitted together like the vertebrae of a spinal column. The mutually facing oblique surfaces 151a and 152a of adjacent insulating disks 15 permit such bending because they rest against one another not in planar fashion but with an acute-angled space left open, and come into contact against one another only after metal tube 13 has been correspondingly curved.

The configuration of the insulating elements is not limited to the geometric conformation of insulating disks 15. For example, insulating disks 15 can also, in the subregion of their disk surfaces, be beveled on only one of the sides facing away from one another or can be rounded on one or both sides. All that is important for the subsequent bending of sheathing tube 13 is that the insulating elements, braced against one another in one subregion, not touch one another in the other subregion within the bracing plane but rather have a clearance from one another that increases toward the outer periphery of the insulating elements. This clearance can be brought about by beveling or rounding on one or both sides. The insulating elements can, however, also be embodied as spheres that rest against one another at a single point, or as spherical caps that are serially arranged in the same direction, so that the one spherical shell is always braced in single-point fashion against the plane of the next spherical cap.

1-21. (canceled)

22. A connecting lead for a sensor, comprising:
a sheathing tube;
at least two electrical conductors extending in the sheathing tube; and
an insulating arrangement that electrically insulates the at least two electrical conductors from one another and from the sheathing tube, wherein the insulating arrangement includes a plurality of insulating elements that are braced against one another and that contain at least two first through holes through each of which one of the at least two electrical conductors is guided.

23. The connecting lead as recited in claim 22, wherein the sensor is for determining a physical property of a measured gas.

24. The connecting lead as recited in claim 22, wherein the sensor is for determining one of an oxygen content and a temperature in an exhaust gas of an internal combustion engine.

25. The connecting lead as recited in claim 22, wherein the insulating elements are braced against one another in one element subregion and have, in another element subregion remaining in a bracing plane, a clearance from one another that increases toward an element periphery.

26. The connecting lead as recited in claim 22, wherein the insulating elements are at least partially braced with their outer periphery in the sheathing tube.

27. The connecting lead as recited in claim 25, wherein:
the insulating elements include insulating disks containing disk surfaces resting against one another, and
at least one of the disk surfaces is beveled toward a disk center.

28. The connecting lead as recited in claim 27, wherein the first through holes are in each insulating disk and are disposed in such a way that their hole axes lie on one diameter line.

29. The connecting lead as recited in claim 28, wherein:
the disk surfaces include side surfaces, and
the bevels in the side surfaces are made in such a way that on each disk surface a parallel surface extending at right angles to the hole axes and an oblique surface angled with respect thereto are present, each extending as far as the diameter line defining the hole axes of the first through holes.

30. The connecting lead as recited in claim 29, wherein the disks have on their peripheral surfaces a respective flat surface that extends parallel to the diameter line defining the hole axes of the first through holes.

31. The connecting lead as recited in claim 30, further comprising:
a round spring rod, wherein:
the disks each have second through opening,
the second through openings in the disks rest against one another and are mutually aligned, and
the round spring rod is retained in axially nondisplaceable fashion in the sheathing tube and is guided through the second through openings of the disks.

32. The connecting lead as recited in claim 31, wherein the second through openings of the disks are disposed in a region of the straight surfaces of the disks, with a radial spacing from the diameter line defining the hole axes of the first through holes.

33. The connecting lead as recited in claim 32, wherein:
each disk includes, on the disk surfaces facing away from one another, respectively, a concave indentation and a convex protrusion that are embodied in such a way that the indentations and protrusions of the disks resting against one another engage conformingly into one another.

34. The connecting lead as recited in claim 33, wherein the concave indentations and the convex protrusions respectively surround entrance openings and exit openings of the first through holes.

35. The connecting lead as recited in claim 34, wherein the two outer ones of the disks resting against one another are braced axially in the sheathing tube.

36. The connecting lead as recited in claim 35, further comprising:
a seal element by which the bracing of the outer insulating disk at the one end of the sheathing tube is accomplished, the seal element being made of electrically insulating material and pressed into the sheathing tube.

37. The connecting lead as recited in claim 36, wherein the seal element has on its periphery circumferential sealing lips, axially spaced apart from one another, that press against an inner wall of the sheathing tube.

38. The connecting lead as recited in claim 37, further comprising:
a further insulating element by which the bracing of the outer insulating disk at another end of the sheathing tube is accomplished, the further insulating element bracing against the sheathing tube.

39. The connecting lead as recited in claim 38, wherein the further insulating element includes an axial through orifice, aligned with the second through openings in the disks, through which the round spring rod is guided.

40. The connecting lead as recited in claim 39, wherein:
the other end of the sheathing tube is closed off by at least one end disk, made of electrically insulating material and resting axially against the insulating element, that exhibits a disposition of third through holes corresponding to a desired contacting pattern of the electrical conductors emerging from the sheathing tube, and
fourth through orifices are introduced into the insulating element for passage of the electrical conductors,
the fourth through orifices create a transition from the exit openings of the first through holes in the adjacent insulating disk to the entrance openings of the third through holes in the adjacent end disk.

41. The connecting lead as recited in claim 40, wherein the round spring rod is braced axially against the end disk and against the seal element.

42. The connecting lead as recited in claim 41, wherein the sheathing tube is crimped over onto the end disk.

43. The connecting lead as recited in claim 42, further comprising:
a connecting cable to which the electrical conductors are each joined by ultrasonic welding, wherein the seal element surrounds the joining points and the connecting cable is guided out of the seal element.

44. The connecting lead as recited in claim 43, wherein the insulating element and the at least one end disk have, on disk surfaces facing away from another, respectively a concave indentation and a convex protrusion having an identical geometry adapted to the indentations and protrusions on the other disks.
